(19) United States
(12) Patent Application Publication
(10) Pub. No.: US 2004/0126814 A1
(43) Pub. Date: Jul. 1, 2004

(54) Sensor having molecularly imprinted polymers

(76) Inventors: Waheguru Pal Singh, College Station, TX (US); Anthony Giletto, College Station, TX (US); Jinseong Kim, College Station, TX (US); Craig Andrews, College Station, TX (US); Jerome Brown, College Station, TX (US); Brian Watkins, College Station, TX (US); Anuncia Gonzalez-Martin, San Jose, CA (US)

Correspondence Address: STREETS & STEELE, 13831 NORTHWEST FREEWAY, SUITE 355, HOUSTON, TX, 77040, US

(21) Appl. No.: 10/663,228

(22) Filed: Sep. 16, 2003

Related U.S. Application Data
(63) Continuation-in-part of application No. 10/379,386, filed on Mar. 4, 2003.
Application No. 10/379,386 is a continuation-in-part of application No. 09/642,796, filed on Aug. 21, 2000, now Pat. No. 6,582,971.

(51) Int. Cl. G01N 33/53; C12M 1/34

(52) U.S. Cl. 435/7.1; 435/287.2; 205/777.5

(57) ABSTRACT

A sensor for detecting a target analyte using a molecularly imprinted polymer (MIP) that has been imprinted with the target analyte. The MIP may be used as a working electrode in electrochemical impedance spectroscopy by either coating a substrate or being pressed into a disk. The senor may also use the MIP for detecting a target analyte through other electrochemical

This patent application is a continuation-in-part of U.S. patent application Ser. Number 10/379,386, which was a continuation-in-part of U.S. application Ser. No. 09/642,796 filed Aug. 21, 2000, now U.S. Pat. No. 6,582,971.

### BACKGROUND OF THE INVENTION

1. Field of the Invention

This invention relates to electronic sensors, and more particularly to electronic sensors used for identifying and separating large molecules.

2. Background of the Related Art

Since the onset of modern medicine, physicians and scientists have sought to understand the physiological changes that occur in human beings during various disorders and diseases. Of prime interest are the changes that occur in various parameters of a patient's blood and urine because these changes are useful diagnostic tools. Cardiovascular condition, hematological/immunological status and toxicological indicators may be determined by real-time, point-of-care biochemical analysis of bio-samples, such as blood and urine, providing critical information to doctors that may be used for treating patients. For example, blood myoglobin, blood creatine phosphokinase MB, and blood Tryponin I are indicators of myocardial infarction, and urinary β-1-macroglobulin is an indicator of kidney glomerulus damage that results from exposure to toxic substances.

Proteins are routinely used as biomarkers for diagnosis, treatment and assessment of a number of physiological functions of the body, and may be used for analysis of bio samples taken from humans and animals in space. Current biomolecular recognition elements for large proteins and other high molecular weight biological compounds rely on fragile biomolecules, such as antibodies, nucleic acid probes, enzymes, and/or other receptors, for recognition of the target analyte. These recognition elements are expensive to prepare, especially in large quantities, and are physically and chemically fragile. Furthermore, sensors and separation media that use these fragile recognition elements have only limited utility because of their expense and fragility.

Responding to the need for an inexpensive, synthetic, and rugged recognition element to replace fragile antibodies, nucleic acids, enzymes, and other bioreceptors, a method of preparing a molecularly imprinted polymer was disclosed in U.S. Pat. No. 6,582,971, hereby fully incorporated by reference. Molecular imprinting of polymers is a process that co-polymerizes functional monomers with cross-linking monomers/polymers in the presence of a target analyte that acts as a molecular template. Before polymerization, the functional monomers either form a complex with the template via non-covalent interactions, or are covalently coupled, forming a polymerizable derivative of the template. After polymerization, the functional groups of the monomers are held in position by the highly cross-linked polymeric structure. Subsequent removal of the template by solvent extraction or chemical cleavage reveals binding sites that are complementary in size and shape to the analyte. In this way, a molecular memory is introduced in the polymer, which becomes capable of rebinding the analyte with very high specificity.

Originally, molecularly imprinted polymers (MIP) were employed as stationary phases in HPLC, notably for chiral separation. Subsequently, their use has been extended to other analytical techniques such as thin layer chromatography, capillary electrochromatography, solid-phase extraction, and immunoassay type binding assays. The binding sites often have affinities and selectivities approaching those of antibody-antigen systems, and have been dubbed antibody-binding mimics. These mimics display clear advantages over real antibodies for sensor technology. Because of their highly cross-linked nature, MIPs are intrinsically stable and robust, facilitating their application in extreme environments, such as in the presence of acids, bases, metal ions, organic solvents, or at high temperatures and pressures. Moreover, MIPs can be inexpensively produced and stored in a dry or wet state at room temperature for long periods of time.

What is lacking in the field of bio sample analysis are methods and apparatus that may be used in a variety of special environments. It would be beneficial to have methods and apparatus that could perform analysis of bio samples quickly and effectively, even in extreme environments, such as the microgravity environment of space flights. It would also be beneficial to have methods and apparatus that could quickly and efficiently provide analysis of bio samples taken from humans or other animals, both quantitatively and qualitatively.

### SUMMARY OF THE INVENTION

The present invention provides sensors having molecularly imprinted polymers (MIP) for detecting a target analyte. One preferred molecularly imprinted polymer suitable for each of the disclosed sensors is formed by a method comprising dissolving a print molecule and a monomer in a first phase and dissolving a host polymer in a second phase, wherein the first and second phases are different phases and selected from an aqueous phase and an organic phase; preparing an emulsion of the aqueous phase and the organic phase; polymerizing the monomer to form a polymer composite with the host polymer along in interface between the first and second phases; and removing the print molecule from the composite. Other alternate molecularly imprinted polymers may also be used in the sensors of the present invention.

In one embodiment, the sensor includes a working electrode having a molecularly imprinted polymer that has been imprinted with the target analyte. The sensor further includes a counter and reference electrode, a potentiostat and a phase analyzer. The counter electrode and the reference electrode, which optionally may be combined into one electrode, is connected to a potentiostat that is connected to the phase analyzer. This device utilizes electrochemical impedance spectroscopy to determine the concentration of the target analyte in a sample.

The counter electrode may be selected from materials such as platinum, gold, titanium, silver, copper, carbon, graphite and combinations thereof and may take the form of mesh, wire, flag, sheet, bar and combinations thereof. The reference electrode may be selected from a silver/silver chloride electrode, a platinized platinum electrode, a calomel electrode. Alternatively, the reference electrode may be selected from materials such as platinum, gold, silver, copper, titanium and combinations thereof.

To provide computing needs and to display the results to the user, the sensor also includes a processor and display, such as found in a personal digital assistant, a personal computer, such as a desktop computer or a laptop computer, or other similar device.

The working electrode may be a compressed disk of MIP or alternatively, the MIP may be coated upon a substrate. The substrate may be electrically conducting and may be formed of material selected from, for example, copper, aluminum, platinum, titanium, gold, and combinations thereof. Alternatively, the substrate may be non-conducting and plated with an electrically conductive material. Acceptable non-conducting substrate materials include silica, silica wafers, alumina, alumina plates, polyimide, Teflon, cyanate ester, Kevlar, printed circuit board, glass epoxy board FR-4, glass epoxy board FR-5 and combinations thereof. These non-conducting substrates are plated with electrically conductive material such as, for example, gold, aluminum, silver, platinum, copper and combinations thereof.

The working, counter and reference electrodes are submersed in an electrolyte. The electrolyte may be selected from a buffer solution, deionized water, an organic solution, a biological solution, phosphate buffer solution, Tris buffer solution, blood, urine, blood plasma and combinations thereof.

In one embodiment of the present invention, the sensor may be a small handheld device having a chip assembly comprising one or more working electrodes either formed from or coated with a molecularly imprinted polymer that is imprinted with the target analyte. The sensor may further include one or more reagent reservoirs, one or more sample reservoirs and a fluid channel in fluid communication with the reagent and sample reservoirs. The sensor further includes reference and counter electrodes, a potentiostat and a phase analyzer connected together as disclosed above.

The present invention further includes a method for determining the presence of a target analyte, comprising placing a working electrode, counter electrode and reference electrode in fluid communication with an electrolyte and an analyte; exerting an electrical potential between the working electrode and the reference electrode; perturbing the electrical potential; and measuring the impedance between the electrolyte and the working electrode with the electrical potential constant, wherein the working electrode comprises a molecularly imprinted polymer. The measured impedance may then be matched to a known impedance for a concentration of the target molecule, wherein impedance is a function of the concentration of the target molecule.

The foregoing and other objects, features and advantages of the invention will be apparent from the following more particular description of a preferred embodiment of the invention, as illustrated in the accompanying drawing wherein like reference numbers represent like parts of the invention.

### BRIEF DESCRIPTION OF THE DRAWINGS

FIGS. 1 A-B are schematic diagrams of exemplary systems that may be used in accordance with the present invention.

FIG. 2 illustrates how target molecules may bind to a molecularly imprinted polymer.

FIG. 3 is a conceptual diagram of the interfacial polymerization process and the resulting molecularly imprinted composite.

FIG. 4 is a perspective view of an exemplary analyzer in accordance with the present invention.

FIGS. 5A-C are perspective and top views of the chip assembly and its components.

FIGS. 6A-C are graphs showing electrochemical impedance signals in a Nyquist plot and Bode plot for varying myoglobin concentrations.

FIG. 7 is a graph of average impedance amplitudes as a function of myoglobin concentration in a range of frequencies with an MIP coated working electrode.

FIG. 8 is a graph of average impedance amplitudes as a function of myoglobin concentration in a range of frequencies with a non-imprinted polymer coated working electrode.

FIG. 9 is a schematic of a miniaturized exemplary electrochemical cell in accordance with the present invention.

FIG. 10 is a graph of average impedance amplitudes as a function of myoglobin concentration in a range of frequencies with the miniaturized electrochemical cell in accordance with the present invention.

FIG. 11 is a graph of average impedance amplitude as a function of CPK concentration over a range of frequencies with MIP-coated and non-imprinted polymer coated working electrodes.

### DETAILED DESCRIPTION OF THE INVENTION

The present invention provides a sensor for detecting a target analyte. In one embodiment, the sensor comprises an electrochemical cell that includes a working electrode having a molecularly imprinted polymer. The molecularly imprinted polymer (MIP) is imprinted with the target analyte, which may be a molecule, bacterium or virus. The sensor further comprises a counter electrode, a reference electrode, a potentiostat, a phase analyzer and a data processor. The electrochemical cell of the sensor comprises the counter electrode, the reference electrode and the working electrode, which further comprises the molecularly imprinted polymer. These electrodes are immersed in an electrolyte and connected to the potentiostat, which is coupled to the phase analyzer and the data processor.

The reference electrode may be an electrode selected from a silver/silver chloride electrode, a platinized platinum wire electrode or a miniaturized calomel electrode. The reference electrode may be made of other materials known to those having ordinary skill in the art, such as gold, silver, platinum, copper or titanium in a variety of forms, including mesh, wire, and flat surface forms such as sheets or bars. The counter electrode may be selected from a variety of metals or other conducting materials, such as platinum, gold, titanium, copper, carbon, or graphite in a variety of forms, including mesh, wire, and flat surface forms such as sheets or bars. The electrolyte may be selected from a variety of liquid solutions, such as a buffer solution (e.g., phosphate buffer solution or Tris buffer solution), deionized water, organic solutions and biological fluids, such as plasma, urine, and whole blood.

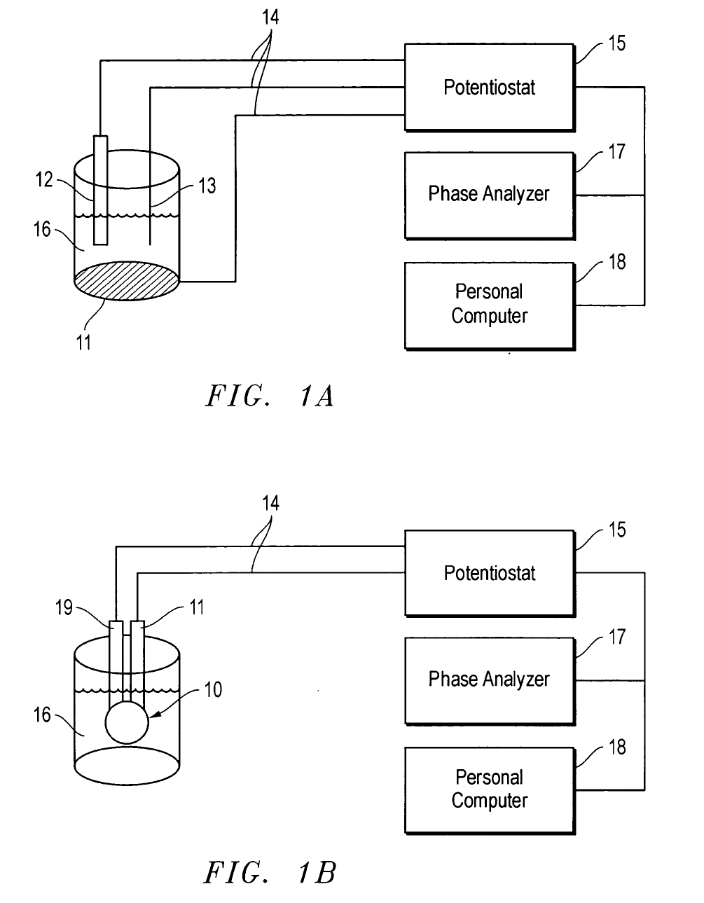

The sensor of one embodiment uses electrochemical impedance spectroscopy (EIS) to determine whether the target molecule exists in a sample and if so, at what concentration. EIS is a well-developed branch of alternating current (AC) electrical theory that describes the current response of a measured electrochemical cell to a perturbation made by an alternating voltage signal of small amplitude. FIG. 1A is a schematic of a system that may be used with EIS in accordance with the present invention. The MIP-coated working electrode 11, the counter electrode 12 and the reference electrode 13 are all immersed in an electrolyte 16. Wires 14 attach each of the electrodes to a potentiostat 15. The potentiostat 15 measures the potential between the working electrode 11 and the reference electrode 13, and adjusts a current through the counter electrode 12 to maintain a pre-determined set point potential between the working electrode 11 and the reference electrode 13. A small AC voltage, represented as a sinusoidal function (dV) and typically having a magnitude of between about 5 mV and about 500 mV, is generated by the phase analyzer 17 and superimposed to the DC voltage being measured by the potentiostat 15 across the working electrode 11 and the reference electrode 13. A perturbation of the current results, which is represented as a sinusoidal function (d1) between the working electrode 11 and the counter electrode 12 and measured by the phase analyzer 17 as a function of frequency. The current response is the change in the flow of the current through the counter electrode 12 made by the potentiostat 15 to maintain the setpoint potential between the reference electrode 13 and the working electrode 11.

FIG. 1B is a schematic of an alternative system that may be used with EIS in accordance with the present invention. An MIP pressed disk 10 connects the working electrode 11 to the combined counter and reference electrode 19, and all are immersed in an electrolyte 16. Wires 14 attach the electrodes 11, 19 to a potentiostat 15. The potentiostat 15 measures the applied open circuit potential between the working electrode 11 and the combined counter and reference electrode 19. A small AC voltage, represented as a sinusoidal function (dV) and typically having a magnitude of between about 5 mV and about 500 mV, is generated by the phase analyzer 17 and superimposed to the DC voltage being measured by the potentiostat 15 across the working electrode 11 and the combined counter and reference electrode 19. A perturbation of the current results, which is represented as a sinusoidal function (d1) between the working electrode 11 and the combined counter and reference electrode 19 and measured by the phase analyzer 17 as a function of frequency. The current response is the change in the flow of the current through the counter electrode 12 made by the potentiostat 15 to maintain the set point potential between the combined counter and reference electrode 19 and the working electrode 11.

The frequency of the voltage perturbation may be varied in the range from mHz to MHz and current response to the voltage perturbation may be measured. The impedance, Z, of the electrode/electrolyte interface, measured in Ohms, is defined as:

$$Z = E / I = \frac{{E}^{′} + {E}^{″} j}{{I}^{′} + {I}^{″} j} . \qquad ( Eq . 1 )$$

where E′ and E″, and I′ and I″ are real and imaginary components of the sinusoidal voltage and sinusoidal current, respectively, represented as vectors, and j=√−1. The vector notation is an easy to understand presentation of the measured values of the real (in-phase) and the imaginary (out-of-phase) components of the impedance. The in-phase component exhibits resistive properties, while the out-of-phase component represents capacitive properties of the measured system. The absolute magnitude of the impedance |Z|, defines the length of the impedance vector:

|Z|=\sqrt{Z′²+Z″²} (Eq. 2)

Another parameter measured in the impedance is the phase angle between the applied sinusoidal voltage and sinusoidal current:

$$θ = \frac{{Z}^{″}}{{Z}^{′}} \qquad ( Eq . 3 )$$

In the simplest form, these measured values, impedance and phase shift, are presented in a Bode plot as a function of the frequency of the voltage applied. The Nyquist plot, often used in conjunction with the Bode plot, plots the resistive properties (Z′) against the capacitive properties (Z″). A Bode plot, a Nyquist plot or a combination of the two plots can be used to evaluate the response in the system. Each impedance system consists of a number of capacitive and resistive components connected in series or in parallel. By choosing a proper frequency range, capacitive and resistive components of a particular process at the solid/electrolyte interface can be separated and measured with increased sensitivity.

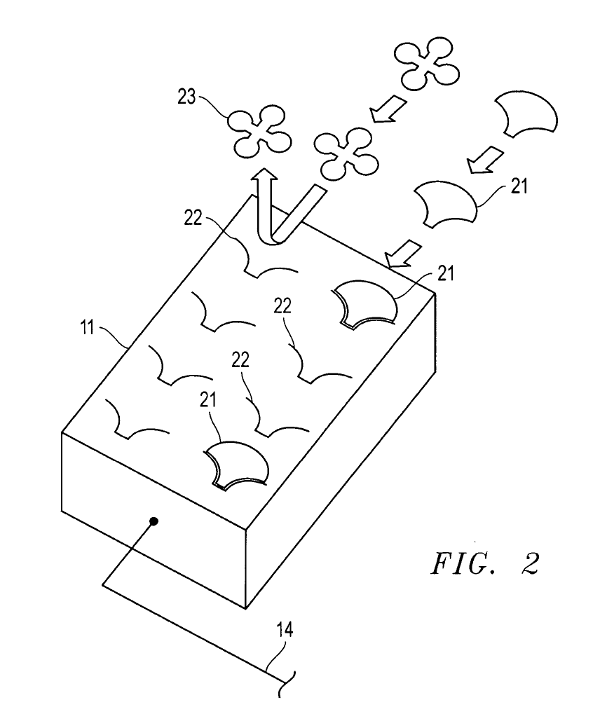

When the working electrode is coated with a molecularly imprinted polymer (MIP), the impedance between the working electrode and the counter electrode changes as the target analyte attach themselves to the MIP. FIG. 2 illustrates how target molecules may attach themselves to the working electrode. The working electrode 11 is coated with the MIP. Target molecule recognition sites 22 are imprinted into the MIP that coats the working electrode 11. A sample to be analyzed contains target molecules 21 as well as non-target molecules 23. A sample that is to be analyzed for the target molecules is added to the electrolyte in which the working electrode 11 is immersed. Target molecules 21 are the complimentary shape and size to the target molecule recognition sites 22, and therefore bind themselves to these recognition sites 22. On the other hand, the non-target molecules 23 have a non-complimentary shape and size to the target molecule recognition sites 22 and therefore, cannot bind themselves to the recognition sites 22. As target molecules 21 bind themselves to the working electrode 11, the impedance between the working electrode 11 and the counter electrode 12 changes as a function of the number of target molecules 21 bound to the surface of the working electrode 11. This impedance change is measured and analyzed through electrochemical impedance spectroscopy as discussed above.

The change in the measured impedance between the working electrode and the counter electrode correlates to the concentration of the target molecule in the sample that is being analyzed. The MIP sensor responds to changes in amplitude as a function of target protein concentration and does not respond to the addition of non-target molecules for which there are no binding sites available. This may be seen in Table 1 and Table 2.

TABLE 1
Impedance Amplitude Measured with a Myoglobin MIP

| | 0 ppm | | | 20 ppm HRP |
|---|---|---|---|---|
| | HRP | 20 ppm HRP | 30 ppm HRP | 20 ppm myoglobin |
| 10 Hz | 165 Ω | 165 Ω | 165 Ω | 195 Ω |
| 100 Hz | 145 Ω | 145 Ω | 145 Ω | 180 Ω |
| 1000 Hz | 140 Ω | 140 Ω | 140 Ω | 175 Ω |

TABLE 2
Impedance Amplitude Measured with a Myoglobin MIP in Urine

| | Urine | PBS | Myoglobin |
|---|---|---|---|
| 10 Hz | 50 Ω | 50 Ω | 62 Ω |
| 100 Hz | 23 Ω | 23 Ω | 24 Ω |
| 1000 Hz | 15 Ω | 15 Ω | 19 Ω |

Table 1 shows the impedance amplitude measured with a working electrode coated with a myoglobin MIP and exposed to horseradish peroxidase (HRP) with concentrations ranging from 0 to 30 ppm. No change in the measured impedance was detected when only HRP was added. However, when 20 ppm of myoglobin was added along with 15 ppm of HRP, a jump in impedance amplitude was measured as shown in Table 1.

Table 2 shows the impedance amplitude measured with a working electrode coated with a myoglobin MIP and exposed to urine, urine spiked with phosphate buffer solution (PBS) and urine spiked with PBS containing myoglobin. Again, no change in the measured impedance occurs between the base line observed with urine and the urine spiked with PBS. However, when the urine was spiked with myoglobin, the impedance amplitude jumped as measured and as shown in Table 2.

Therefore, as shown by the results of Tables 1 and 2, the MIP sensor is quite selective and specific for the target molecule with which the MIP was imprinted.

A preferred molecularly imprinted polymer and its method of making is disclosed in U.S. Pat. No. 6,582,971, which is hereby fully incorporated by reference. The disclosed process produces the molecularly imprinted polymer by the interfacial polymerization of a monomer in the presence of the print molecule and a host polymer. Advantageously, since the polymerization occurs at the interface between an organic solvent and an aqueous solution, the print molecule can be disposed in the phase that allows the print molecule to remain in its native configuration. The choice of a host polymer and a monomer to be polymerized can be varied to enhance the specificity of the composite toward the target molecule to be imprinted.

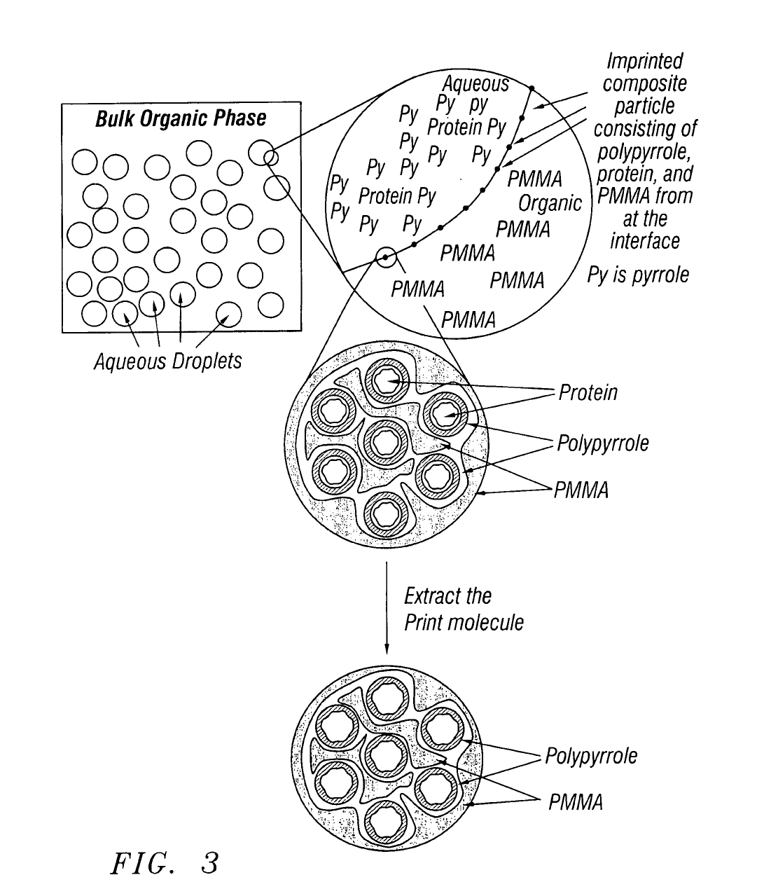

The disclosed interfacial polymerization process includes an organic liquid phase and an aqueous liquid phase, in which typically five components are dissolved: the host polymer, monomers to be polymerized, the compound to be imprinted (the print molecule), the surfactant, and the polymerization initiator. FIG. 3 is a conceptual diagram of the interfacial polymerization process resulting in the molecularly imprinted composite. As shown in FIG. 3, the host polymer is disposed in the organic phase and the monomer and print molecule are disposed in the aqueous phase. This distribution is not required. In fact, some biomolecules, for example lipids, glycolipids, and membrane proteins, may loose their three dimensional structure when dissolved in aqueous solutions. Therefore, for imprinting some biomolecules it may be appropriate to dissolve the print molecule and the monomer into the organic phase and a host polymer into the aqueous phase to yield the most specific imprinted composite. The most preferred phase for the host polymer, the monomer to be polymerized, and the print molecule should be considered on a case-by-case basis, though typically the molecular imprinting occurs through an interfacial polymerization processes having the print molecule and the monomers in the same phase and the host polymer in a different phase. The method of imprinting the molecularly imprinted polymers may vary and the description of forming molecularly imprinted polymers contained herein is not meant to limit the present invention.

Again referring to FIG. 3, the host polymer (such as polymethylmethacrylate) is soluble in the organic phase (such as chloroform) while the molecule to be imprinted (such as horseradish peroxidase) and the monomer (such as pyrrole) are soluble in the aqueous phase. The monomer forms specific and non-specific interactions with the print molecule, forming the basis of the specific binding sites in the final product. Polymerization of the monomers begins through the addition of an initiator (such as ferric chloride). Although the monomer is soluble in the aqueous phase, the host polymer is not. Therefore, as the polymer extends in length, it accumulates at the interface between phases as it partitions from the aqueous phase to the organic phase. The print molecule also accumulates at the interface through its interaction with the polymer. The resulting polymer and print molecule are trapped in this configuration by the host polymer in the organic phase, providing composite particles consisting of host polymer, newly formed polymer, and print molecules. The composite particles are recovered and the print molecules are extracted to yield the polymer composite having binding sites that are specific for the print molecule.

An emulsion of the MIP may be prepared for coating the working electrode of the sensor. The MIP-emulsion is prepared by mixing a host polymer, monomers to be polymerized and the print molecule as disclosed above. A quantity of the MIP-emulsion is then mixed with a photo-reactive initiator, such as a variety of silver salts selected from, for example, silver nitrate, silver tosylate, or from organic compounds, such as benzophenone. The MIP-emulsion is mixed with the photo-reactive initiator at a mole ratio of emulsion to initiator of between about 1000 to 1 and about 1 to 1. Preferably the ratio is between about 50 to 1 and about 10 to 1. For example, the photo-reactive initiator may be prepared by dissolving 75 mg of silver nitrate in 1 mL of acetonitrile and 1 mL of freshly distilled pyrrole. This initiator solution would be added to the MIP-emulsion and exposed to UV light to polymerize the polymer. The emulsion is applied using a variety of methods, such as spin coating, spray coating, and dip coating onto the surface of the working electrode substrate. The working electrode substrate may be any electrically conductive material such as copper, aluminum, platinum, titanium and gold. Alternatively, the substrate may be a non-conductive material that has been plated with a conductive material. The non-conductive material may be selected from a variety of silica wafers, alumina plates and polymeric surfaces such as Teflon, Kevlar, polyimide, and cyanate ester. Preferably, the working electrode substrate may be made from printed circuit boards, glass epoxy FR-4, glass epoxy FR-5, or combinations thereof. The conductive material used to plate the non-conductive material may be selected, for example, from gold, silver, aluminum, and copper.

After the MIP-emulsion has been applied to the working electrode substrate, the electrodes are exposed to UV light for photopolymerization to occur. The magnitude of UV power and duration of time for complete polymerization is dependant upon the thickness of the deposited MIP-emulsion on the substrate. The photopolymerization causes the MIP coating to adhere to the surface of the electrode substrate. The polymer-coated electrodes should be thoroughly washed with, for example, methanol, other organic solvents, buffer solutions, acids, bases, or enzyme solutions and then with deionized water before being thoroughly air-dried.

The diameter and thickness of the MIP electrode is proportional to the expected detection levels of the target analyte. The method used to imprint the polymer with the target molecule, as well as other physical characteristics of the polymer, determines the number of binding sites on the MIP that are available to the target analyte. The size of the MIP working electrode is adjusted based upon the number of binding sites that are available on the MIP so that maximum changes in impedance may be observed when the target analyte binds to the electrode. For example, if a protein in plasma occurs in the range of 10 μg/mL-45 μg/mL and 1 mL of plasma is to be analyzed, then a suitable MIP electrode may be formed that is capable of binding 50 μg of the protein to cause a maximum change in impedance. An MIP electrode having binding sites to accommodate 50 μg of the protein may have a working electrode that is 3 mm in diameter and 100 μm thick if the MIP has binding sites sufficient to bind 1 μg of protein per 14 μm of MIP.

Alternatively, a pressed disc made of MIP material may form the working electrode. The MIP may be formed into a pressed disc by first grinding dry MIP to a fine powder. A portion of the powdered MIP is placed in a shaped die and pressed with about 5000 pounds of force at room temperature to form an MIP disc. The MIP may be pressed into a variety of shapes, sizes and thicknesses as may be appropriate for the given MIP and application. One suitable size is a disc that is about 13-mm in diameter and about 0.5 mm thick. It should be noted that the polymerized coating of MIP onto the working electrode substrate and the use of the MIP as a pressed disc or other shape are merely shown as examples of how the MIP may be used as an electrode. These examples are not meant to limit the form of the MIP that may be used as a working electrode.

All sensors have a finite response time. An MIP sensor that incorporates AC impedance analysis of a target analyte binding to the imprinted sites of the MIP working electrode may typically perform the AC impedance analysis in less than 1 minute after exposing the MIP to the target analyte. This quick response is due to the rapid mass transfer of the target analyte to the MIP electrode. However, the time required for binding of the target analyte to the working electrode prior to impedance analysis is dependent on the analyte being studied. The transfer rate of the target analyte to the MIP working electrode determines the period required before the impedance analysis may begin, and this period may range from seconds to minutes.

There are a wide range of techniques available for measuring AC impedance spectra with complexities ranging from the Wheatstone bridge to the latest in high-speed digital signal processors that provide mixing, decimation, and signal analysis. The selection of a particular AC impedance measurement technique for a given application involves compromise and the balancing of critical parameters such as power consumption, frequency range, accuracy, measurement time, and signal/noise.

The AC impedance spectra may include the frequency response of the sample measured at several discrete frequencies and the sample characterization is the result of a unique ‘fingerprint’ of the impedances. Following the technique described above, the simultaneous measurement of multiple frequencies may be accomplished by using a single waveform, which is the summation of the individual reference frequencies. The sample is perturbed with this summation waveform and the sample response multiplied and correlated with each of the original individual reference frequencies. While this technique reduces sample time by allowing all frequencies of interest to be measured simultaneously, it substantially increases the signal processing requirements since each frequency is typically over-sampled at a minimum rate of four times (2×for each of the real and imaginary components) the highest frequency. This Nyquist over sampling results in an increased onboard processing capability requirement and a subsequent increase in power consumption.

In some cases, the 10 Hz response provides enhanced sensitivity over the higher frequency measurements. However, potentially more important than a single frequency response is the collective response of the imprinted polymer over a wide range of discrete frequencies or across a continuous frequency. Measurement of a sample as the sampling frequency is swept is commonly referred to as a Frequency Response Analysis (FRA). From this evaluation, the sampling frequency requirements may be selected from single frequency, multiple discrete frequencies, or frequency sweep. Preferably, as long as acceptable target molecule concentration sensitivity and selectivity are obtained, the minimum acceptable number of sampling frequencies should be employed.

The electronic hardware for the AC impedance system may be divided into four major subsystems: the reference circuit, the input amplifier, the demodulator, and the dc filter. If multiple frequencies are simultaneously measured, each frequency typically has its own real and imaginary demodulators and filters. Furthermore, to automate the impedance measurement, some form of automatic correlation is provided, typically in the form of a digital processor. A preferred choice for high frequency, multiple-response analysis is field programmable gate arrays (FPGA), having one of the highest processing power. Alternatively, high-end systems comprising digital signal processors (DSP) and function specific, single package waveform generators/demodulators/decimators that are specifically designed and marketed for electrochemical impedance spectroscopy applications may be used. However, for the MIP application with frequencies ranging from 10 Hz to 1 kHz, these high-end solutions are disadvantageous because of their size and power requirements. At the extreme opposite end of the spectra from these high-end solutions is the complete analog solution that uses various voltage-to-current converters, integrators, error signal generators, function generators, and amplifiers. These analog systems are plagued with various calibration issues, are inherently inflexible in their use, and have poor signal-to-noise ratios. Therefore, while the high-end solutions and the complete analogue system may be used in accordance with the present invention, these solutions are not preferred.

A preferred solution is a combination of high quality input amplifiers for signal gain followed by a wide analog-to-digital converter. This digitized signal may then be analyzed by a processor (either microprocessor or digital signal processor), which also provides the correlation function. The ‘arbitrary’ waveform, sweeping, or pure sine wave reference signal may also be generated by the processor using internal tables and a high-speed digital-to-analog converter.

A graphical user interface may be a part of the digital processor and may be selected from a personal computer, a laptop computer, a personal digital assistant (PDA) or a mainframe computer. Preferably, a PDA using the Microsoft Windows operating system may be used. A PDA system may be equipped with a serial interface as well as proprietary interfaces that may be used with various flash cards, modems, cameras, etc. Using a PDA having the Windows operating system simplifies transferring programs between systems operating with the popular Windows operating systems. Another benefit of using a PDA as the operating platform is the integration of the complete user interface with other program applications, such as spreadsheet programs. This integration allows data to be easily entered, viewed, and transferred to other computers while minimizing development of proprietary software and hardware.

The impedance system, comprising the electrodes, potentiostat, and phase analyzer, may be connected directly to the PDA or other digital processor. Through the conventional PDA character recognition and menu driven user interface, the user may select a patients name and/or record number, select the assay to be performed, and view the resulting data. This data may be appended to an existing record and compared to historical data, trends, and baseline data. Using existing programs known to those having ordinary skill in the art, this information may be transferred to other computers or digital processors using a serial link, docking station, or infrared link.

Alternatively, if a user interface is not required, communications through an electronics interface between the sensor and a digital processor may be provided through a standard serial port or universal serial bus (USB). In this case, communications drivers and sample programs for communication and data transfer between the instrument and the digital processor become necessary. Optionally, a minimum user interface may be provided as a basic graphical display that is capable of displaying the equipment status and the result of the most recent analysis. Flexible keys adjacent to the display may be used to input fundamental commands such as selection of an assay or scrolling through a menu tree.

Advantageously, an array of MIP working electrodes may be used within a sensor to determine the concentrations and identity of multiple target molecules rapidly and simultaneously. Any of the materials that are suitable for constructing a single working electrode are suitable for constructing a multiple electrode for use in a sensor that analyzes a sample for multiple target molecules. For example, an array of MIP working electrodes may be formed by first cutting copper plated printed circuit board material to an appropriate size. The copper surface may then be etched by using standard etching techniques and by masking the areas that are not to be etched, to form a number of areas of un-etched copper of uniform or desired size. A different MIP, each having been imprinted with a different target analyte, may be deposited on each area so that each of the areas target a different analyte in a sample being analyzed.

FIG. 4 is a perspective view of an exemplary analyzer in accordance with the present invention. As shown in FIG. 4, the sensor may be reduced to a hand-held system comprising a PDA 54 and a custom docking station 51. In addition to providing docking for the PDA 54, the docking station 51 further provides power to the PDA 54 when it is docked and data transmission to and from the PDA 54 for analysis. The docking station 51 further provides housing for the components (not shown) for the electrochemical impedance spectroscopy analysis, such as the phase analyzer and potentiostat. The docking station 51 is adapted for insertion of the chip assembly 52, which may be inserted into the PDA in a manner similar to the insertion of a memory chip into a conventional PDA. The chip assembly 52 may be snapped into place, preferably fitting into the slot in only one direction, thereby making the chip assembly 52 very easy to install. The chip assembly 52 includes reagent reservoirs 61 and sample port 62. Buttons 56 on the face of the PDA 54 are used for user input and a reject button 53 releases the chip assembly 52 from the docking station 54.

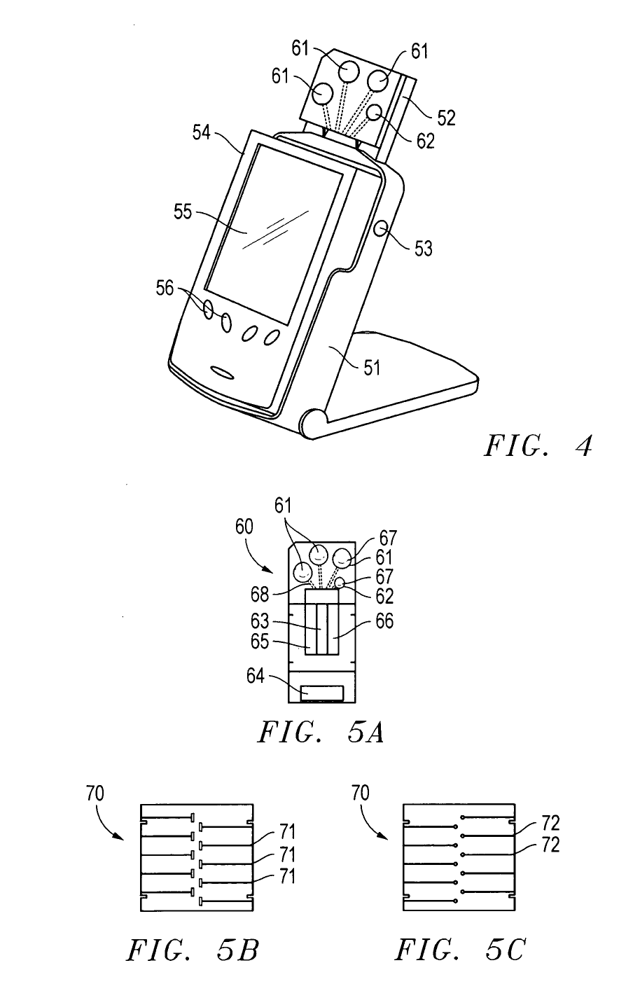

FIGS. 5A-C are perspective and top views of the chip assembly and its components. The chip assembly 52 consists of two parts: the chip 70, which is disposable, and the chip assembly base 60, which may be reusable. The chip base 60, shown in FIG. 5A, contains the sample port 62 and reagent reservoirs 61 and a fluid channel 63 for delivering the reagents and sample to the chip 70. A reference electrode 65 and a counter electrode 66 are in fluid communication with the fluid channel 63. Also included is the waste reservoir 64.

The chip 70 contains the MIP coated electrodes 71 and the etched conductor lines 72. FIG. 5B shows the front of the chip 70 and FIG. 5C shows the back of the chip 70. The exemplary chip shown in FIGS. 5B and 5C includes ten MIP-coated electrodes 71 and conductor lines 72. Each of the MIP-coated electrodes 71, which are contacted with the sample and reagents, are made by coating the working electrode 71 with a molecularly imprinted polymer that is imprinted with the target analyte to be identified by the MIP coated working electrode 71. The conductor lines 72 are on the back of the chip 70 as shown in FIG. 5C. The chip 70 is clipped onto the back of the chip assembly base 60. Reagents and electrolyte contained in the reservoirs 61 are released with the bio-sample contained within the sample reservoir 62 into the fluid channel 63. The fluids released into the channel 63 contact the working electrodes 71 that are coated with MIP's and the reference electrode 65 and the counter electrode 66. Optionally, the reference electrode and the counter electrode may be included on the chip. The chip assembly 52 is then inserted into the base station 51, containing the phase analyzer and digital processor hardware necessary to conduct the electrochemical impedance spectroscopy analysis.

Optionally, a disposable permeable sheath (not shown) may be contained within the waste reservoir 64. The sheath may be filled with a fluid absorbent polymer powder. The sample and reagents may flow into the disposable sheath, and then discarded after the sample has been analyzed. This sheath is a disposable item that should be replaced after each use and rinse cycle.

The front of the chip 70, shown in FIG. 5C, includes the exposed MIP-coated working electrodes 71 that face the fluid channel 63 when the chip 70 is clipped onto the chip assembly base 60. When the chip is clipped onto the base 60, a tight seal is provided between the channel 63 and the chip 70. The chip assembly base 60 may be made of thermoplastics having high strength to weight ratios such as PEI/PEEK. The covers over the reagent, absorbent and chip areas may also be made of the same material. These polymers lend themselves well to mass production methods such as injection molding. The mating portions of the base 60 material and the chip 70 may be over molded with a thin layer of medical grade silicone to provide a good seal when the chip 70 is clipped onto the base 60. The septums 67 covering the reagent reservoirs 61 and the sample reservoir 62 may be formed of an elastomer, such as silicone, over molded onto the chip assembly base 60. Optionally, the reagent pathways 68 into the fluid channel 63 may have an embedded check valve (not shown) built into reagent pathways 68 to prevent fluid from cross contaminating the sample reservoir 62.

When the reagent septum 67 is pressed, fluid is forced from the reagent reservoir 61, through the channel 63, over the MIP-coated working electrodes 71, and finally into the waste reservoir 64 that contains a polymer absorbent. After use, the reagent reservoirs 61 optionally may be refilled with the appropriate reagents by injecting reagents through the septums 67. When fresh reagent is injected, the chip 70 should be removed and the sample should be introduced until a small quantity comes out of the channel. This ensures that the channel is completely filled with fluid and that there are no air bubbles. The test sample is introduced into the channel by injecting the sample through the sample septum. The test sample chamber should be thoroughly rinsed with DI water/sterilant after each use to remove any residual sample material that could adversely affect the subsequent tests. Once the various reagents have passed over the MIP-coated working electrode, the chip is introduced into the docking station and a customized program on the PDA is executed to collect the signal data. The data may be further analyzed on the PDA itself, with the results reported on the LCD screen. Optionally, the data may be exported to a personal computer for storage or further analysis. Once the test is completed, the chip should be discarded along with the polymer absorbent. However, the base piece can be cleaned and reused. The system can operate on battery power, is hand held and portable allowing it to be used in a variety of environments.

While the preceding discussion has focused primarily upon the use of molecularly imprinted polymer based sensors using electrochemical impedance spectroscopy for quantitatively and qualitatively determining the existence of various target molecules, the invention is not limited only to the use of electrochemical impedance spectroscopy. Other detection methods may be coupled with the use of molecularly imprinted polymers for use in sensors including various electrochemical, optical, acoustical and microscopic techniques.

Additional electrochemical methods of detection that may be used with a molecularly imprinted polymer surface include resistiometric, amperometric, and/or potentiometric methods (e.g. cyclic voltammetry) of analyte detection, monitoring changes in resistance, current, and/or potential as a result of target molecules binding to the MIP surface. Changes in resistance, current, and/or potential are directly proportional to the analyte concentration. A wafer of molecularly imprinted polymer may be exposed to the analyte to allow the analyte to bind to the wafer, and then the current, resistance and/or potential of the wafer may be measured. The change in the current, resistance and/or potential between the wafer that is free of bound target molecules and the wafer that has bound target molecules provides a measurement of the concentration of the target molecule in the analyte.

Therefore, regarding electrochemical detection methods, if measuring a change in current, an embodiment of the present invention includes an MIP working electrode, a counter electrode, a reference electrode and a potentiostat to hold potential constant and measure a change in current. If measuring a change in potential, an embodiment of the present invention includes an MIP working electrode, a counter electrode, a reference electrode, and a galvanostat to hold current constant and measure changes in potential. If measuring a change in resistance, an embodiment of the present invention includes an MIP working electrode, a counter electrode, and an ohmmeter to measure resistance. The counter and reference electrodes in these embodiments may be selected from those previously disclosed herein. The analyte/reagent used in these embodiments may provide the connection between the electrodes in embodiments having two or three electrodes or the MIP may provide the connection in a two electrode system. A personal digital assistant, personal computer or similar device may be used to display the results of the analysis and provide the computing means.

If the analyte contains a target molecule of interest having a functional group or functional molecule that has reduction/oxidation potential, then electrochemical techniques such as cyclic voltammetry can be applied to determine analyte concentration. The MIP binding sites serve as a trap for the target analyte providing electrochemical detection of the target analyte without interference from other outside molecules. In the case of cyclic voltammetry monitoring the reduction peak, or oxidation peak, or both peaks of the analyte once trapped in of MIP will correspond directly to the amount of analyte present in a given sample. The hardware requirement for this embodiment include an MIP working electrode, and counter and reference electrodes as disclosed previously as well as a potentiostat to measure the current as particular voltages are applied to the system. A personal digital assistant, personal computer or similar device may be used to display the results of the analysis and provide the computing means.

Another embodiment of the present invention utilizes an electrochemical transduction method that includes applying a field effect transistor (FET) as a signal transducer for the MIP's. In this embodiment, an FET is fabricated with MIP to function as a selective dielectric material. As with other chemical sensors using FETs as the signal transducer, the MIP fabricated FET (MIPFET) responds to changes to the surface of the applied selective material. By selectively targeting a specific molecule for FET surface adhesion, the signal response is specific for that target analyte. The binding of target analyte to the surface of the MIP modulates the surface-energy of the MIPs work function, thereby inducing a change in field, measured by monitoring the change in drain/source current with constant applied gate potential. This can also be achieved by holding the current constant and monitoring the fluctuation in potential across the drain/source electrodes.

If the target analyte of interest contains an optically active label (e.g., acrylodan-labeled albumins) or is optically active itself (e.g., hemoglobin, myoglobin, etc.), a reflectance based analysis can measure the amount of bound analyte on the MIP surface. The direct surface reflectance of the MIP changes as a function of analyte concentration that is monitored via UV-VIS, IR, or Raman absorption or emission spectroscopy. In this embodiment, the reflected wavelength of interest is measured on the MIP surface before and after the addition of test/sample solutions. The measurements taken before the addition of the test/sample solutions provide a background level. Differences in the measured reflectance before and after the addition of the test/sample solutions are a function of the amount of bound analyte on the MIP surface. The MIP surface may be provided as a wafer of MIPs or may be a surface having MIPs bound thereon as previously disclosed. The light source may be provided as, for example, an LED and the reflectance may be measured with, for example, a photodiode. Alternatively, other light sources and measuring devices may be used as is well known to those having ordinary skill in the art.

Reflectance can also be measured from the backside of an MIP coating using a thin film of MIP coated onto the tip of a fiber-optic cable or onto the surface of other optically transparent material, such as a quartz crystal. Using TIR (totally internal reflectance) spectroscopy, surface changes to the MIP after exposure to the target molecules may be determined by measuring spectroscopic changes to the evanescent wave that propagates through the MIP material. These changes may include, for example, wavelength shift and changes in intensity. At low angles, total internal reflection results when light propagating within a dense medium, such as the quartz, reaches an interface with a less dense medium, such as the MIP coating. Although the light is fully reflected, an evanescent field is generated that extends beyond the interface and into the MIP coating. Typically, the penetration depth, or the thickness of evanescent field, is in the range of half the wavelength of the light. The evanescent field provides the surface selectivity of TIR. Only target molecules that selectively bind to the MIP on the surface become excited and therefore emit. The target analyte concentration is proportional to the absorption or emission changes in the TIR. As with the other disclosed embodiments, a personal digital assistant, personal computer or other similar device may be used for displaying the results and performing the required computing needs. A preferred embodiment includes an optic fiber as a light source, MIP deposited on the surface of the optic fiber and a detector, such as a photodiode for measuring the changes to the evanescent wave.

Yet another embodiment of the present invention includes using the MIP for detecting target molecules as a trapping site. In this embodiment, the MIP serves as a capture antibody that selectively binds a target biomolecule, such as a protein. The method includes adding a first secondary antibody, for example, a biotinylated reporter, to the MIP/biomolecule solution to form a sandwich complex between the first secondary antibody and the MIP/biomolecule. Then, a second fluorescently-tagged secondary antibody, such as streptavidin in this example, is added to selectively bind to the first secondary antibody (e.g., the biotinylated reporter) and fluorescence is emitted. The emitted fluorescence is proportional to the amount of protein captured by the MIP and can be quantitatively measured by a fluorimeter. This embodiment includes a sensor having an MIP preferably formed into a wafer and a fluorimeter. Additionally included is a palm pilot, personal computer or other similar device for displaying the results and for providing necessary computation means.

The conducting molecularly imprinted polymers can be further used as a sensor recognition element by methods incorporating piezoelectricity transducers and bulk acoustic wave resonance spectroscopy. The conducting MIPs can be coated onto the surface of piezoelectric quartz crystals (PQC). The selective absorption process at the interface has an affect on the oscillation of the MIP coated PQC and gives responses to changes upon analyte loading. Target analyte binding to the MIP coated PQC proportionally effects its oscillation and the signal transduced by monitoring and recording the resulting changes in resonant frequency using any frequency counting device.

An MIP based sensor using imprinted conducting polymer does not require the use of the MIP as the transducer. The MIP may act as a trap for a targeted analyte and a secondary transducer may be used to actually detect the analyte. A variety of the electrochemical, optical, and acoustical techniques disclosed herein can be incorporated as a secondary transducer of the analyte. For example, in the case of Enzyme Linked Immunosorbent Assays (ELISA), the MIP can serve as a primary artificial antibody for a targeted protein and secondary antibodies can bind to the trapped protein and allow for an absorption or emission spectroscopic technique to be performed. A variety of other detection techniques can use the ability of the MIP to isolate the target analyte for analysis. In the case of proteins, the use of Matrix Assisted Laser Desorption Ionization Spectroscopy (MALDI) is successful in determining the presence of a targeted protein. However, for analysis by MALDI, typically the protein is first isolated by using a separation gel. MIP can provide the necessary trapping that analysis by MALDI requires to determine presence of the targeted protein.

### EXAMPLE 1

### Molecularly Imprinted Polymer Synthesis

A solution of 0.8 g polymethylmethacrylate (PMMA), having an average molecular weight of 15,000, in 10 mL CHCl₃ was added to the aqueous phase consisting of 200 mg sodium lauryl sulfate and 0.5 mL pyrrole dissolved in 2.0 mL deionized water with stirring. When making the MIP, 50 mg of horse myoglobin was also added to the aqueous phase. After the two phases formed an MIP-emulsion upon rapid stirring, FeCl₃ solution (0.1 g/mL water) was added drop-wise (1 drop/sec) with external cooling provided by an ice/water bath. After 4 hours, the reaction was quenched with 100 mL of methanol. The mixture was stirred for an additional hour. MIP was vacuum filtered and washed 5 times with 50 mL water and 5 times with 50 mL methanol. The MIP was then dried overnight at a reduced pressure (30 Hg) under 35° C., ground by hand using a mortar and pestle, and sifted using a standard sieve to a size less than 106 μm. This washing and drying procedure was then repeated before the polymer was stored in capped 15 mL falcon tubes at room temperature. Non-imprinted polymers (NIPs) were synthesized using the same procedure without the addition of a target molecule.

### EXAMPLE 2

### Coating Electrodes with Imprinted Polymers

To make the photopolymerization (PP) solution, 0.075 g AgNO₃ was dissolved in 1 mL acetonitrile, and 1 mL freshly distilled pyrrole was added into the solution in a 3 mL amber vial. Then, 0.2 mL of PP solution and 0.1, 0.2, or 0.3 mL of MIP-emulsion were mixed together and applied to three different electrode surfaces, fully covering the surfaces with the mixture. The MIP-emulsion was formed as described in Example 1. The electrodes were then exposed to UV light (mercury lamp, λₘₐₓ 260 nm, approx. 50 to 80 W) for 5 minutes to induce the photopolymerization. The electrodes, now coated with photopolymerized MIP's, were washed 5 times with 1 mL 40% methanol in 5 mM phosphate buffer solution pH 3, then rinsed 5 times with 1 mL deionized water, then washed 5 times with 1 mL 40% methanol in 5 mM phosphate buffer solution pH 7, and rinsed 5 times with 1 mL deionized water, thereby removing all the horse myoglobin from the MIP structure and leaving empty sites for subsequent target molecule attachment. The MIP-covered electrode was air dried at room temperature.

### EXAMPLE 3

### Concentration of Target Molecule Changes Impedance

Using a system as shown in FIG. 1A, having a working electrode formed as in Example 2 and using myoglobin as the target molecule, the electrodes were immersed in a 5.5 mL phosphate buffer solution pH 7.4. Electrochemical impedance signals for myoglobin-MIP on the working electrode were collected. Impedance signals were collected at open circuit potential, at an AC voltage of 10 mV, and scanning the frequency from 10 KHz to 1 Hz.

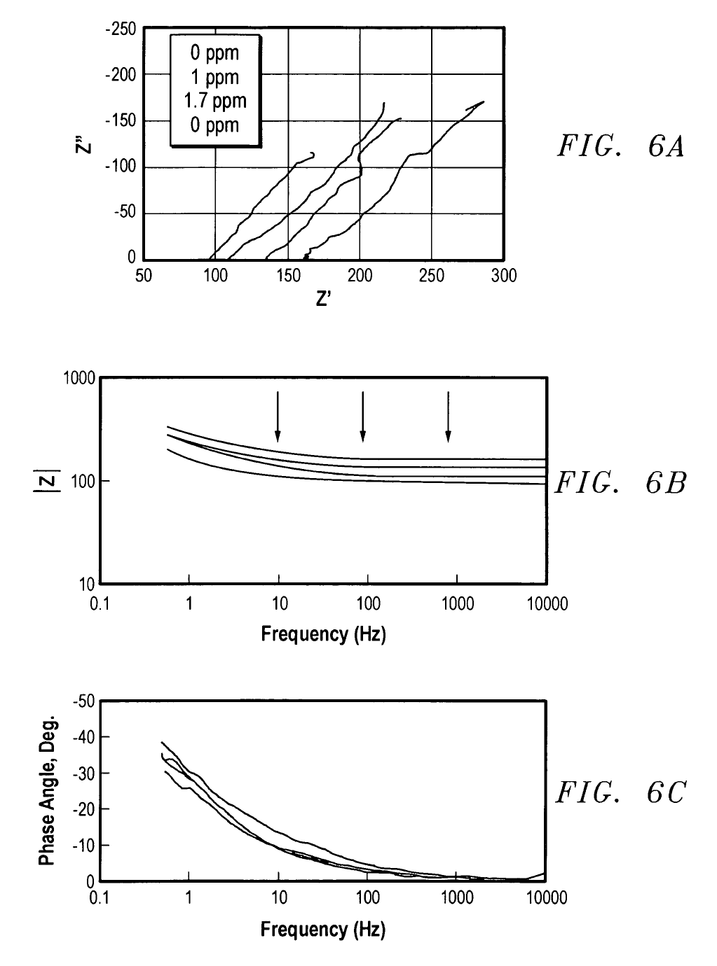

First, electrochemical impedance was measured with 0 ppm myoglobin in the phosphate buffer solution as background impedance. Then, 1.5 mL of 5 ppm myoglobin was added to the phosphate buffer solution to make a 1 ppm myoglobin solution and the electrochemical impedance was measured again. More of the 5 ppm myoglobin solution was added to the electrochemical cell to make a solution of 1.7 ppm myoglobin and impedance was again measured. The results are shown in FIGS. 6A-C. FIG. 6A shows the complex plot (imaginary versus real components of impedance) and FIGS. 6B-C show the Bode plot (amplitude and phase shift versus frequency) as a function of the myoglobin concentration (0, 1, 1.7, 0 ppm in the sequence) in the phosphate buffer solution. As the myoglobin concentration increases, the data points on the complex plot moved away from the origin of the plot. When the electrochemical system was flushed with a fresh PBS, the signals moved back to the origin of the plot.

### EXAMPLE 4

### Concentration of Target Molecule Changes Impedance at Single Frequency

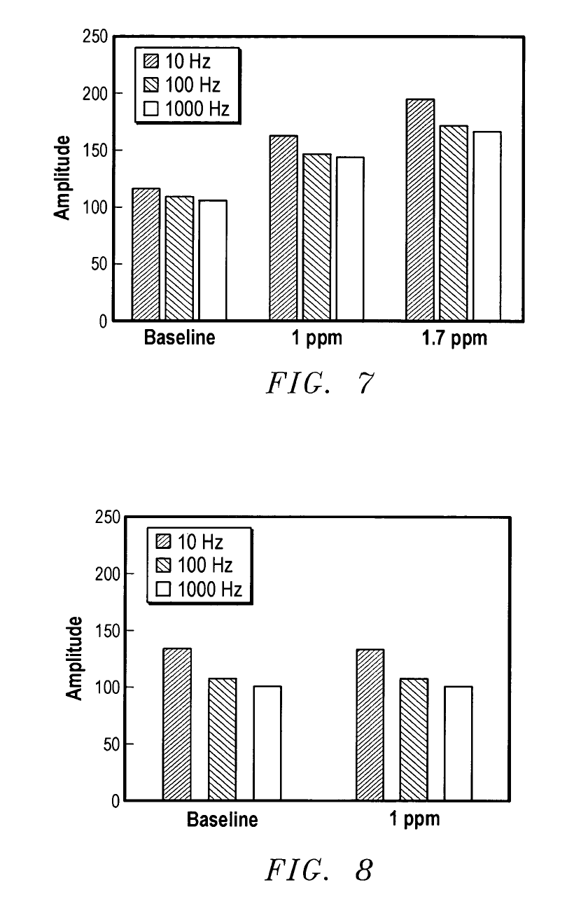

Although complex impedance plots contain important information about the dynamics of the target molecule/MIP/electrode interaction, it is simpler and quicker, and therefore more desirable, to have a single signal showing the response of the MIP sensor to the presence of the target molecule. Using a system as shown in FIG. 1-A, a working electrode coated with an MIP imprinted with myoglobin, a reference electrode and a counter electrode are immersed in a phosphate buffer solution (PBS) electrolyte. Three frequencies (10, 100, 1000 Hz) were selected to study the impedance signal as a function of myoglobin concentration (0 ppm myoglobin, 1 ppm myoglobin and 1.7 ppm myoglobin). FIG. 7 shows that the average of the impedance amplitudes changed substantially as a function of myoglobin concentration in the phosphate buffer solution over the range of the selected frequencies. To prove that the signal variation was due to the presence of myoglobin in the phosphate buffer solution, the same experiment was repeated using a non-imprinted polymer (NIP) sensor. FIG. 8 shows that no changes in impedance were observed when the non-imprinted polymer coated electrode was used. There were no impedance changes shown in FIG. 8 because the non-imprinted polymer lacked the binding sites for the target molecule to bind with the polymer.

### EXAMPLE 5

### Impedance Independent of Sample Volume or Electrode Surface Area

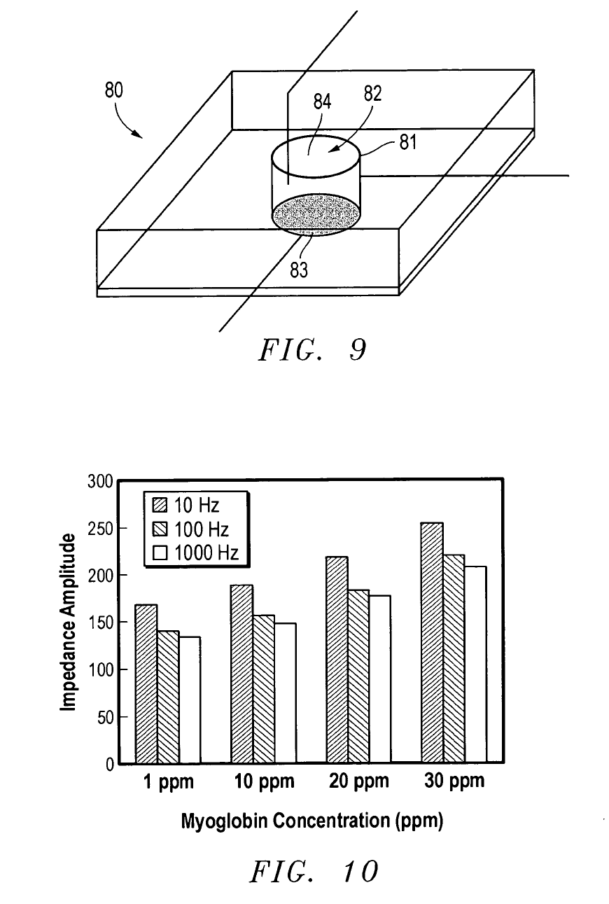

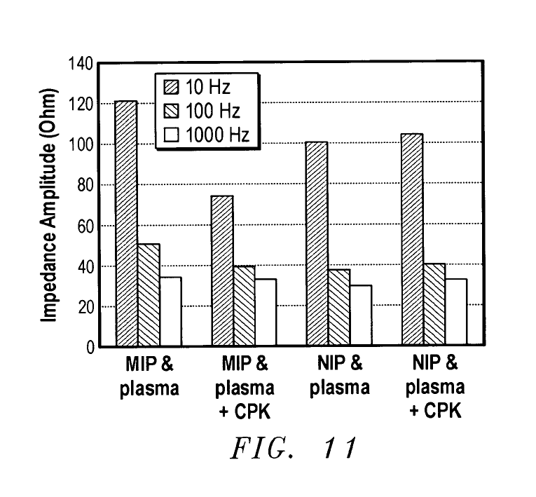

In Examples 3 and 4, liquid volumes of about 7 mL of phosphate buffer solution were used as electrolyte for the immersion of the electrodes and the sample volume. FIG. 9 is a schematic of a miniaturized exemplary electrochemical cell in accordance with the present invention. This miniaturized electrochemical cell 80 was formed of polydimethylsiloxane (PDMS). The well 82 held about 0.7 mL of the combined sample volume and the phosphate buffer solution. The MIP coated working electrode 83 had a surface area of about 1 cm². By comparison, the surface areas of the working electrodes in Examples 3 and 4 were about 1.7 cm². A ring of Pt gauze was used as a counter electrode 81 and a silver/silver chloride electrode was used as the reference electrode 84. The change in the surface area of the electrode from 1.72 cm² to 1 cm² and the change in the amount of sample from 1.5 mL to 0.7 mL had no effect on the resulting impedance measurements. FIG. 10 is a graph showing that the average impedance amplitude at 10, 100, and 1000 Hz changed as a function of myoglobin concentration using the miniaturized cell. The impedance amplitude was found to be independent of the liquid volume or the surface area of the working electrode and was found to depend only upon the myoglobin concentration, which was kept in the physiological range of myoglobin concentrations desirable (1 to 15 mg/l) in human urine. The range of the concentrations in this experiment was kept between about 10 and 30 ppm myoglobin.

### EXAMPLE 6

### Impedance Changes as a Function of Concentration of CPK in Plasma

Using a system as shown in FIG. 1-A with a working electrode formed as in Example 2, but using creatine phosphokinase (CPK) as the imprinted molecule on the MIP, and with the electrodes immersed in a 5.5 mL phosphate buffer solution (10 mM K₃PO₄) pH 7, electrochemical impedance signals for CPK-MIP on the working electrode were collected. First, 0.7 mL of blank plasma solution was introduced to the phosphate buffer solution and the impedance results were recorded at 10, 100, and 1000 Hz. Then, 0.1 mL of the plasma was removed and replaced with 0.1 mL of 10 ppm CPK spiked plasma solution, making the overall concentration of CPK 1 ppm. Impedance was again recorded. The same experiment was then repeated using a working electrode coated with a non-imprinted polymer. The results are shown in FIG. 11. The addition of the CPK spiked plasma resulted in an approximate 38% decrease of impedance amplitude at 10 Hz, and approximately a 22% decrease at 100 Hz. Under the same conditions, the non-imprinted polymer coated electrode showed a 4% and 8% increase of the impedance amplitude at 10 and 100 Hz respectively.

It will be understood that certain combinations and sub-combinations of the invention are of utility and may be employed without reference to other features in sub-combinations. This is contemplated by and is within the scope of the present invention. As many possible embodiments may be made of this invention without departing from the spirit and scope thereof, it is to be understood that all matters hereinabove set forth or shown in the accompanying drawings are to be interpreted as illustrative and not in a limiting sense.

What is claimed is:

1. A sensor for detecting a target analyte, comprising:
a working electrode comprising a molecularly imprinted polymer, wherein the molecularly imprinted polymer is imprinted with the target analyte.

2. The sensor of claim 1, further comprising:
a counter electrode;
a reference electrode;
a potentiostat; and
a phase analyzer, wherein the working electrode, the counter electrode and the reference electrode are connected to the potentiostat and wherein the potentiostat is connected to the phase analyzer.

3. The sensor of claim 2, wherein the material for the counter electrode is selected from platinum, gold, titanium, silver, copper and combinations thereof.

4. The sensor of claim 3, wherein the counter electrode has a form selected from mesh, wire, flag, sheet, bar and combinations thereof.

5. The sensor of claim 2, wherein the material for the counter electrode is selected from carbon, graphite and combinations thereof.

6. The sensor of claim 5, wherein the counter electrode has a form selected from mesh, wire, flag, sheet, bar and combinations thereof.

7. The sensor of claim 2, wherein the reference electrode is selected from a silver/silver chloride electrode, a platinized platinum electrode, and a calomel electrode.

8. The sensor of claim 2, wherein material for the reference electrode is selected from platinum, gold, silver, copper, titanium and combinations thereof.

9. The sensor of claim 2, wherein the reference electrode is a miniaturized saturated calomel electrode.

10. The sensor of claim 2, further comprising:
a processor, wherein the processor is connected to the phase analyzer.

11. The sensor of claim 10, wherein the processor is selected from an analogue processor and a digital processor.

12. The sensor of claim 10, wherein the processor performs electrochemical impedance spectroscopy analysis on a fluid contacting the working electrode.

13. The sensor of claim 10, wherein the processor is selected from a desktop computer, a laptop computer, a personal digital assistant, and combinations thereof.

14. The sensor of claim 1, wherein the working electrode further comprises a substrate.

15. The sensor of claim 14, wherein the molecularly imprinted polymer coats the substrate.

16. The sensor of claim 14, wherein the substrate is electrically conductive.

17. The sensor of claim 16, wherein the substrate is selected from copper, aluminum, platinum, titanium, gold, and combinations thereof.

18. The sensor of claim 16, wherein the substrate is a metal.

19. The sensor of claim 14, wherein the substrate is electrically non-conductive and wherein the substrate is plated with an electrically conductive material.

20. The sensor of claim 19, wherein material of the non-conductive substrate is selected from silica, silica wafers, alumina, alumina plates, polyimide, Teflon, cyanate ester, Kevlar, and combinations thereof.

21. The sensor of claim 19, wherein the material of the non-conductive substrate is selected from printed circuit board, glass epoxy board FR-4, glass epoxy board FR-5, and combinations thereof.

22. The sensor of claim 19, wherein the electrically conductive material is selected from gold, aluminum, silver, platinum, copper and combinations thereof.

23. The sensor of claim 1, wherein the working electrode is pressed molecularly imprinted polymer.

24. The sensor of claim 2, further comprising:
an electrolyte, wherein the working electrode, the reference electrode and the counter electrode are immersed in the electrolyte.

25. The sensor or claim 24, wherein the reference electrode and the counter electrode is a common electrode.

26. The sensor of claim 24, wherein the electrolyte is selected from a buffer solution, deionized water, an organic solution, a biological solution, and combinations thereof.

27. The sensor of claim 24, wherein the electrolyte is selected from phosphate buffer solution, Tris buffer solution, and combinations thereof.

28. The sensor of claim 24, wherein the electrolyte is selected from blood, urine, blood plasma, and combinations thereof.

29. The sensor of claim 1, wherein the target analyte is a biomolecule.

30. The sensor of claim 29, wherein the biomolecule is selected from a protein, a glycolipid, a lipid, a polysaccharide, and a polynucleotide.

31. The sensor of claim 1, wherein the target analyte is selected from a virus and a bacterium.

32. The sensor of claim 1, wherein the molecularly imprinted polymer is prepared by a method comprising:
dissolving a print molecule and a monomer in a first phase and dissolving a host polymer in a second phase, wherein the first and second phases are different phases and selected from an aqueous phase and an organic phase;
preparing an emulsion of the aqueous phase and the organic phase;
polymerizing the monomer to form a polymer composite with the host polymer along in interface between the first and second phases; and
removing the print molecule from the composite.

33. A sensor for detecting a target analyte, comprising:
a chip assembly comprising one or more working electrodes, wherein the one or more working electrodes comprise a molecularly imprinted polymer, and wherein the molecularly imprinted polymer is imprinted with the target analyte.

34. The sensor of claim 33, wherein the chip assembly further comprises:
one or more reagent reservoirs;
one or more sample reservoirs; and
a fluid channel in fluid communication with the one or more reagent and sample reservoirs, and with the one or more working electrodes.

35. The sensor of claim 34, further comprising:
a reference electrode;
a counter electrode;
a potentiostat; and
a phase analyzer, wherein the reference electrode and the counter electrode are in fluid communication with the fluid channel, and wherein the working electrode, the counter electrode and the reference electrode are in electrical communication with the potentiostat, and wherein the potentiostat is in electrical communication with the phase analyzer.

36. The sensor of claim 35, wherein the counter electrode is a metal.

37. The sensor of claim 35, wherein the material for the counter electrode is selected from platinum, gold, titanium, copper, silver and combinations thereof.

38. The sensor of claim 35, wherein the reference electrode is a metal.

39. The sensor of claim 35, wherein the reference electrode is selected from platinum, gold, titanium, copper, silver and combinations thereof.

40. The sensor of claim 35, wherein the reference electrode and the counter electrode are formed on a substrate.

41. The sensor of claim 35, wherein the one or more working electrodes are formed on a substrate.

42. The sensor of claim 41, wherein the substrate is electrically non-conductive, wherein the substrate is plated with an electrically conductive material, and wherein the one or more working electrodes comprise the electrically conductive material.

43. The sensor of claim 42, wherein the electrically conductive material is a metal.

44. The sensor of claim 42, wherein the electrically conductive material is selected from gold, platinum, copper, silver, titanium, aluminum and combinations thereof.

45. The sensor of claim 41, wherein the substrate is a material selected from silica, silica wafers, alumina, alumina plates, polyimide, Teflon, cyanate ester, Kevlar, and combinations thereof.

46. The sensor of claim 41, wherein the substrate is a material selected from printed circuit board, glass epoxy board FR-4, glass epoxy board FR-5, and combinations thereof.

47. The sensor of claim 33, further comprising:
a digital processor; and
means for conducting electrochemical impedance spectroscopy on a fluid contacting the one or more working electrodes, wherein the digital processor is in electrical communication with the means for conducting electrochemical impedance spectroscopy.

48. The sensor of claim 47, wherein the means for conducting electrochemical impedance spectroscopy comprises:
one or more input amplifiers;
a wide analog-to-digital converter.

49. The sensor of claim 47, wherein the digital processor is selected from a personal computer, a main frame computer, a personal digital assistant and a laptop computer.

50. The sensor of claim 34, further comprising:
an electrolyte, wherein the one or more working electrodes, the reference electrode and the counter electrode are immersed in the electrolyte.

51. The sensor of claim 50, wherein the electrolyte is selected from a buffer solution, deionized water, an organic solution, a biological solution, and combinations thereof.

52. The sensor of claim 51, wherein the electrolyte is selected from blood, urine, blood plasma, and combinations thereof.

53. The sensor of claim 33, wherein the target analyte is a biomolecule.

54. The sensor of claim 53, wherein the biomolecule is selected from a protein, a glycolipid, a lipid, a polysaccharide, and a polynucleotide.

55. The sensor of claim 33, wherein the target analyte is selected from a virus and a bacterium.

56. The sensor of claim 33, wherein the molecularly imprinted polymer is prepared by a method comprising:
dissolving a print molecule and a monomer in a first phase and dissolving a host polymer in a second phase, wherein the first and second phases are different phases and selected from an aqueous phase and an organic phase;
preparing an emulsion of the aqueous phase and the organic phase;
polymerizing the monomer to form a polymer composite with the host polymer along in interface between the first and second phases; and
removing the print molecule from the composite.

57. A method for determining the presence of a target molecule in an analyte, comprising:
placing a working electrode, counter electrode and reference electrode in fluid communication with an electrolyte and an analyte;
exerting an electrical potential between the working electrode and the reference electrode;
perturbing the electrical potential;
measuring the impedance between the electrolyte and the working electrode with the electrical potential constant, wherein the working electrode comprises a molecularly imprinted polymer.

58. The method of claim 57, further comprising:
matching the measured impedance to a known impedance for a concentration of the target molecule, wherein impedance is a function of the concentration of the target molecule.

59. The method of claim 57, wherein the working electrode further comprises a substrate.

60. The method of claim 59, wherein the substrate is coated with the molecularly imprinted polymer.
